SEQUENCE LISTING (21 sequences)

SEQ ID NO: 1 - DNA, 567 bp, Artificial sequence
  Synthetic plasmid (Wild-type)
gaattcgcccttaacctttttcaggtaaaaaaaaaaaaaaaaaaaaaaaaaagggttcatgacggaggttgtgaggcgcggatcccaactacatgtgtaacagttccatcgatgtaacagttcctgcatgggcggcgatatctaacagttcctgcatgggcggcggtacccctgcatgggcggcatgaaccggctgcagctgcatgggcggcatgaaccgggagctcgacggaacagctttgaggtgcgtgtcgacacggaacagctttgaggtgcgtaaatagcagaaataaaagaaaagatggtaccgtccacctgtacactatgttcagctgcagctcaaacagcacaaaccaagcgagagctcgctccctccaaaagtggtgctcagacacgtgcctgtcctgggagagaccggcgcacaggtggagatctgcaaacctcgctttgaagaatccaatcttttcttttatttctgctatttgcagtgcttaggtccactctctctcttttcagcagtaagagaaagaggcagaaaaaacctattgaagcttaattaattagctt

SEQ ID NO: 2 - DNA, 547 bp, Artificial sequence
  Synthetic plasmid (Mutant)
gaattcgcccttaacctttttcaggtaaaaaaaagggttcatgacggaggttgtgaggcacggatcccaactacatgtgtaacagtttcatcgatgtaacagttcctgcatgggcagcgatatctaacagttcctgcatgggcgacggtacccctgcatgggcggcatgaactggctgcagctgcatgggcggcatgaaccaggagctcgacggaacagctttgaggtgagtgtcgacacggaacagctttgaggtgcataaatagcagaaataaaagaaaagatggtaccgtccacctgtacactatgtttagctgcagctcaaacagcacaaaccaagtgagagctcactccctccaaaagtggtgcttagacacgtgcctgtcctgggagagactggcgcacaggtggagatctgcaaacctcactttgaagaatccaatcttttcttttatttatgctatttgcagtgcttaggtccactctctcttttcagcagtaagagaaagaggcagaaaaaaactattgaagcttaattaattagctt

SEQ ID NO: 3 - DNA, 20 bp, Artificial sequence
  Synthetic oligonucleotide
ctcaaacagcacaaaccaag

SEQ ID NO: 4 - DNA, 20 bp, Artificial sequence
  Synthetic oligonucleotide
gtgcctgtcctgggagagac

SEQ ID NO: 5 - DNA, 213 bp, Ademomatous polyposis coli (APC) gene 1450-1465 codon
ccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacagaaagtactccagatggattttcttgt

SEQ ID NO: 6 - DNA, 20 bp, Artificial sequence
  Synthetic oligonucleotide
ctcaaacagcacaaaccaag

SEQ ID NO: 7 - DNA, 20 bp, Artificial sequence
  Synthetic oligonucleotide
cacctccaccacctcctcaa

SEQ ID NO: 8 - DNA, 20 bp, Artificial sequence
  Synthetic oligonucleotide
tgcttaggcccactttctct

SEQ ID NO: 9 - DNA, 19 bp, Artificial sequence
  Synthetic oligonucleotide
gtatcagcatctggaagaa

SEQ ID NO: 10 - DNA, 32 bp, Artificial sequence
  Synthetic oligonucleotide
cttttcagcagtaggtgctttatttttaggta

SEQ ID NO: 11 - DNA, 32 bp, Artificial sequence
  Synthetic oligonucleotide
tttcagcagtaggtgctttatttttaggtact

SEQ ID NO: 12 - DNA, 32 bp, Artificial sequence
  Synthetic oligonucleotide
cgagagtacctaaaaataaagcacctactgct

SEQ ID NO: 13 - DNA, 32 bp, Artificial sequence
  Synthetic oligonucleotide
tgagagtacctaaaaataaagcacctactgct

SEQ ID NO: 14 - DNA, 213 bp, Artificial sequence
  Synthetic oligonucleotide
tcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaa

SEQ ID NO: 15 - DNA, 20 bp, Ademomatous polyposis coli (APC) gene 1503-1573 codon
tgagccatttatacagaaag

SEQ ID NO: 16 - DNA, 20 bp, Artificial sequence
  Synthetic oligonucleotide
agagaaagaggcagaaaaaa

SEQ ID NO: 17 - DNA, 21 bp, Artificial sequence
  Synthetic oligonucleotide
tcatcatcatctgaatcatct

SEQ ID NO: 18 - DNA, 26 bp, Artificial sequence
  Synthetic oligonucleotide
ctattgattctgaaaaggacctatta

SEQ ID NO: 19 - DNA, 26 bp, Artificial sequence
  Synthetic oligonucleotide
actattgattctgaaaaggacctatt

SEQ ID NO: 20 - DNA, 33 bp, Artificial sequence
  Synthetic oligonucleotide
cttttcagcagtaagagaaagaggcagaaaaaa

SEQ ID NO: 21 - DNA, 33 bp, Artificial sequence
  Synthetic oligonucleotide
tttcagcagtaagagaaagaggcagaaaaaaac

(19) United States
(12) Patent Application Publication
(10) Pub. No.: US 2008/0241827 A1
(43) Pub. Date: Oct. 2, 2008

(54) Methods For Detecting A Mutant Nucleic Acid

(75) Inventors: Lisa Kann, Burlington, MA (US); Anthony P. Shuber, Mendon, MA (US)

Correspondence Address: GOODWIN PROCTER LLP;PATENT ADMINISTRATOR, EXCHANGE PLACE, BOSTON, MA, 02109-2881, US

(73) Assignee: Exact Sciences Corporation, Marlborough, MA (US)

(21) Appl. No.: 11/596,107

(22) PCT Filed: May 10, 2005
(86) PCT No.: PCT/US05/16518

§ 371 Date: Jan. 31, 2008

Related U.S. Application Data
(60) Provisional application No. 60/569,736, filed on May 10, 2004.
(60) Provisional application No. 60/631,214, filed on Nov. 24, 2004.

(51) Int. Cl. C12Q 1/68; G01N 33/00

(52) U.S. Cl. 435/6; 436/94

(57) ABSTRACT

The invention relates to methods for detection of genomic variation. The invention may be used to analyze nucleic acid sequences to detect low frequency mutations in a sample and/or screen for the presence of a disease.

### TECHNICAL FIELD

The present invention relates generally to methods for detection of genomic variation. More specifically, the invention relates to methods for analyzing nucleic acid sequences to detect mutations present in a sample.

### BACKGROUND OF THE INVENTION

Now that the entire human genome has been sequenced, the next step is to determine genetic variations between individuals. In order to find genetic variations there is a need for more sensitive forms of genetic screening. Additionally, techniques are needed to detect low frequency mutations in heterogeneous samples.

### SUMMARY OF THE INVENTION

Current methods of nucleotide sequencing use a single sequencing reaction containing a mixture of all four terminator nucleotides in the same reaction, where each terminator base is differentially labeled and detected. The signal from an altered sequence present at low concentrations in a sequencing reaction is often masked by the signal of the wild type base at the same location. Variant sequences must be at least about 10% of the DNA being sequenced before their presence can be readily detected. In contrast, methods according to the invention increase the sensitivity of assays to detect nucleic acid alterations that are present at a relatively low level in a sample, especially, e.g., in a heterogeneous sample.

The present invention includes methods of screening nucleic acids for at least one genetic variation through the application of a novel modification of a DNA sequencing reaction. Methods of the invention modify current sequencing reactions such that only one terminator nucleotide (also referred to as a terminating nucleotide, i.e., a nucleotide that terminates an enzymatic primer extension reaction because it cannot be extended by a polymerase enzyme when it is incorporated into a primer extension product), and not all four terminator nucleotides, is provided in a primer extension reaction to allow for single base scanning, which is also referred to herein as single base tracking. The modified reaction is herein referred to as a single base tracking reaction. A single base tracking reaction of the invention may be used to detect the presence, in the nucleic acid region being scanned, of at least one aberrant nucleotide (e.g., a mutation, polymorphism, etc.) corresponding to the terminating nucleotide base being used in the scanning reaction. Aspects of the invention may be used to detect the presence of at least one variant nucleotide in at least one nucleic acid molecule being scanned even in the presence of an excess of nucleic acids that do not contain the variant nucleotide. Such an increased sensitivity has at least several uses. For example, methods according to the invention can be used to screen the human genome, providing for increased sensitivity for detection of low frequency genetic variations (e.g., when screening pools of nucleic acids obtained from a population of individuals in order to identify low frequency genetic variations). Methods according to the invention also can be used to interrogate one or more nucleic acid regions for the presence of at least one genetic variation (e.g., mutation) in at least one nucleic acid molecule in a biological sample that contains many nucleic acid molecules that do not have a genetic variation in the region being scanned. A nucleic acid region being scanned may be at least about 10 bases long, for example about 20 bases, about 50 bases, about 100 bases, about 150 bases, or about 200 bases long. However, in some embodiments the region may be shorter, longer, or of intermediate length.

Methods of the invention can be used to screen for mutations that are predictive or indicative of a disease state. The presence of a mutant nucleic acid molecule in a biological sample may be indicative of a disease such as cancer or pre-cancer (e.g., colon cancer or adenoma). Often, these mutations are present in a sample at a relatively low level, e.g., where the mutation is a somatic mutation in a nucleic acid population obtained from biopsied tissue. Methods according to the invention are more sensitive than current sequencing methods and can detect, in a scanning reaction, the presence of relatively low frequency mutations in a heterogeneous biological sample. According to aspects of the invention, an altered/mutant nucleic acid molecule originating from an adenoma and/or early stage cancer cell (or debris thereof) may be shed into a biological sample along with a large number of corresponding normal nucleic molecules that are shed from normal cells (i.e., non-adenoma and non-cancer cells) that line a lumen from which the biological sample originates or is obtained. An adenoma or early stage cancer is typically small and very few diseased cells (or debris thereof) are shed into the biological sample relative to normal cells (or debris thereof) from the normal tissue surrounding the adenoma or early stage cancer. As a result, altered/mutant nucleic acid molecules indicative of the adenoma or early stage cancer may be very rare relative to the corresponding normal nucleic acid molecules (i.e., nucleic acid molecules with an unaltered or non-mutant sequence from the same region of the genome as the altered/mutant nucleic acid molecule that has the altered/mutant sequence). According to aspects of the invention, indicia of certain later stage cancers also may be present at low frequencies in heterogeneous biological samples. Accordingly, aspects of the invention are useful for disease detection.

According to aspects of the invention, single base tracking reactions increase the sensitivity for detecting low frequency genetic events, at least because signals from bases at any one position in a sequence being scanned are no longer masked by signals from an alternate base in the wild type sequences present at higher concentrations in the sample. Sensitivity for low frequency mutations in a biological sample also may be increased by using certain ratios of extending nucleotides (nucleotides that can be extended in an enzymatic primer extension reaction) to terminating nucleotides; using two or more polymerases with different relative preferences for extending and terminating nucleotides; using certain analytical techniques (e.g., manual techniques, automated techniques, computer-implemented software, or any combination thereof) to quantify one or more signals associated with the incorporation of a known nucleotide (e.g., a labeled terminator nucleotide) at a known position in an extension reaction of the invention; or using any combination of the above techniques. Therefore, methods of the invention may be used to detect the presence of nucleotide sequences with altered residues when compared to a control “wild type” nucleotide sequence, where the nucleic acids with altered sequence make up about 50%, about 25%, about 10%, about 5%, about 4%, about 3%, about 2.5%, about 2%, about 1.5% or especially about 1% of the nucleic acids in the sample being analyzed, or even lower than 1%, for example about 0.5%, or about 0.1% or lower than 0.1% of the nucleic acids in the sample being analyzed.

In a preferred reaction, the terminator nucleotide is labeled. A preferred label is a fluorescent label, although it is within the skill of an artisan to use substitute labels of equal or higher sensitivity in signal detection, and/or equal or lower background signal noise. The DNA single base tracking reaction utilizes sensitive labeling techniques in order that the resulting sequence fragments may be analyzed and, e.g., compared to a known normal control sample to determine whether at least one genetic variation exists between the sample and normal control.

One aspect of the invention includes a method for detecting a difference between two nucleic acids. The method includes extending a first primer complementary to a target nucleic acid in the presence of a first nucleotide and a second nucleotide to produce at least one product. The first nucleotide is at least one deoxynucleotide, and more preferably is a mixture of four deoxynucleotides, namely dATP, dCTP, dGTP and dTTP (“dNTP mixture”) used for the elongation step of the primer extension reaction. The second nucleotide is a terminator nucleotide, preferably includes a detectable label, and has the same base as one of the first deoxynucleotides. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid. Signal differences include the addition of at least one peak, the deletion of at least one peak, or a shift in the position of at least one peak present in the sample as compared to the control.

In another aspect, a scanning reaction may be analyzed for signs of low frequency genetic events (e.g., one or more mutations) without using a comparison to a control or other reference nucleic acid of known sequence. For example, the presence of a mutation at a low frequency may be determined by quantifying the signals obtained for different positions of the primer extension product and determining whether one or more of the signals are present at a low, but statistically significant, level relative to signals for other positions (e.g., at about 10%, about 5%, about 1%, about 0.1% or lower than signals at other positions). Similarly, a corresponding loss of a small, but statistically significant, amount of signal (e.g., a loss of about 10%, about 5%, about 1%, about 0.1% or less than signals at other positions) at a position expected to generate a signal using a different terminator nucleotide may be indicative of (or confirm) the presence of a variant nucleotide at that position for a small number of nucleic acids in the sample being assayed.

The embodiments described above and below can have any or all of the following features. The method may include the step of amplifying a nucleic acid to form the target nucleic acid. The extending step can include extending the primer in the presence of the deoxynucleotides dATP, dCTP, dGTP, and dTTP. The target nucleic acid can be a nucleic acid suspected of containing a mutation. The target nucleotides to be screened in the methods of the invention may be genomic DNA, complementary DNA (cDNA), or RNA. Where the initial sample is RNA, it is preferred that the RNA is converted into DNA prior to further processing. The extending and comparing steps can be repeated. The extending and comparing steps can be conducted two or more times (e.g., at least four times) with the same primer, each time using a different one of adenine (A), cytosine (C), guanine (G) or thymidine (T) for the base of the second “terminating” nucleotide (i.e., each extension reaction contains only one type of extension terminating nucleotide, where the terminating nucleotide may be a dideoxynucleotide or an acyclonucleotide, and the base of the terminating nucleotide is chosen from A, C, G, or T).

A comparison nucleic acid can be a wild type nucleic acid. The signal from the comparison nucleic acid can be determined prior to, at the same time as, or after the signal from the target nucleic acid. The signal can include a fluorescent light emission. Alternatively, the signal results of the control sequence may be obtained from a database of nucleotide sequences. The comparison step may be done manually or by automation.

The methods described above or below can also have any or all of the following features. In certain embodiments, the method includes extending a second primer complementary to the target nucleic acid in the presence of the first nucleotide and the second nucleotide to produce at least one secondary product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one secondary product is the product of a primer extension reaction. The method may also include detecting a signal from the at least one secondary product and comparing the signal from the at least one secondary product with a signal that was generated from a comparison nucleic acid in substantially the same manner as the signal was generated from the target nucleic acid. A difference between the signals indicates at least one difference between the target nucleic acid and the comparison nucleic acid.

The methods described above or below may also include the following features. In one embodiment, a second primer complementary to a strand complementary to the target nucleic acid is extended in the presence of the first nucleotide and the second nucleotide to produce at least one secondary product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one secondary product is the product of a primer extension reaction. The method can then include detecting a signal from the at least one secondary product and comparing the signal from the at least one secondary product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide including a detectable label and a second nucleotide to produce at least one product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer including a detectable label and being complementary to a target nucleic acid in the presence of a first nucleotide and a second nucleotide to produce at least one product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that was generated from a comparison nucleic acid in substantially the same manner as the signal was generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide and a second nucleotide to produce at least one product. The second nucleotide is a terminator nucleotide and includes the same base as the first nucleotide. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a mass of the at least one product and comparing the mass of the at least one product with a mass that is generated from a comparison nucleic acid in substantially the same manner as the mass is generated from the target nucleic acid. A difference between the masses indicates at least one difference between the target nucleic acid and the comparison nucleic acid. Product masses may be determined using electrophoresis, mass spectrometry, or any other suitable technique as the invention is not limited in this respect.

For any of the assays described herein, a single base-tracking primer extension reaction may be cycled 2 or more times, for example between 5 and 40 times, about 30 times (e.g., 28, 29, 30, 31, or 32 times). For any of the assays described herein, PCR primers of between 20 and 50 nucleotides in length may be used. Similarly, primers used in single-base tracking extension reactions may be between about 20 and 50 nucleotides (e.g., about 30 or about 40) in length. However, other numbers of reaction cycles and primer lengths (e.g., longer, shorter, or intermediate lengths) may be used as the invention is not limited in this respect. It should be appreciated that the number of reaction cycles and primer lengths may be adapted to enhance the signal to noise ratio for detecting low frequency mutations in single-base scanning reactions described herein. However, it also should be appreciated that if a positive result is obtained using a single-base scanning reaction, the same assay may be repeated and/or a further assay may be performed in order to confirm the presence of a mutation and/or the frequency of the mutation in the biological sample. These and other aspects, along with advantages and features of the present invention herein disclosed, will become apparent through reference to the following description, the accompanying drawings, and the claims. Furthermore, it is to be understood that the features of various embodiments described herein are not mutually exclusive and can exist in various combinations and permutations. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting. Other features and advantages of the invention will be apparent from the following detailed description and claims.

### BRIEF DESCRIPTION OF THE DRAWINGS

In the drawings, like reference characters generally refer to the same parts throughout different views. Also, the drawings are not necessarily to scale, emphasis instead generally being placed upon illustrating principles of the invention. In the following descriptions, various embodiments of the present invention are described with reference to the following drawings.

FIG. 1 is a normal “wild type” nucleic acid sequence (SEQ ID NO:1) from a plasmid used in an embodiment of the present invention described in Example 1. Tracking Primer D1 (SEQ ID NO:3) is underlined, Tracking Primer G3 (SEQ ID NO:4) is double underlined, and wild type residues for sites mutated in SEQ ID NO:2 are boxed.

FIG. 2 is a mutant nucleic acid sequence (SEQ ID NO:2) from a plasmid used in an embodiment of the present invention described in Example 1. Tracking Primer D1 (SEQ ID NO:3) is underlined, Tracking Primer G3 (SEQ ID NO:4) is double underlined, and mutated sites as compared to SEQ ID NO:1 are boxed. The final mutant is a 2 base deletion compared to the wild type sequence in FIG. 1.

FIG. 3 are signals obtained according to Example 1 showing mutant nucleic acid detection in test samples doped with 4%, 2%, or 1% mutant constructs compared to a normal sample.

FIG. 4 are signals obtained according to Example 1 showing mutant nucleic acid detection in test samples doped with 4%, 2%, or 1% mutant constructs compared to a normal sample.

FIG. 5 are signals obtained according to Example 1 showing mutation detection in test samples doped with 4%, 2%, or 1% mutant constructs compared to a normal sample.

FIG. 6 is a nucleic acid sequence (SEQ ID NO:5, in bold) for a region of the adenomatous polyposis coli (“APC”) gene (codon 1450-1465) according to Example 3. Tracking primer SBE D1 (SEQ ID NO:6) and PCR Primer D For (SEQ ID NO:7) are single and double underlined, respectively, and are shown in the 5′-3′ direction. Tracking primer D3 SBE 1465-5 (SEQ ID NO:8) and PCR Primer D Rev (SEQ ID NO:9) are shown in the 3′-5′ direction.

FIG. 7 are signals obtained according to Example 3, where signals generated from mutant nucleic acid (a 2 base deletion compared to normal) in samples obtained from cancer patients are compared to signals generated from normal nucleic acid samples using labeled “A” terminator nucleotide as the only terminator nucleotide in the reaction. The wild type (“WT”; SEQ ID NO:10) and mutant (“MT”; SEQ ID NO:11) sequences being tracked are shown above the graph, wherein MT is a two (2) base deletion compared to WT.

FIG. 8 shows signals obtained according to Example 3 comparing signals generated from mutant nucleic acid in samples obtained from cancer patients to signals generated from normal nucleic acid samples using labeled “T” terminator nucleotide as the only terminator nucleotide in the reaction. The wild type (“WT”; SEQ ID NO: 12) and mutant (“MT”; SEQ ID NO:13) sequences being tracked are provided, wherein MT is a point mutation.

FIG. 9 is a nucleic acid sequence (SEQ ID NO: 14, in bold) for APC coding region 1503-1573, where the residue being interrogated is at position 1554. PCR primer Q1 For (SEQ ID NO:15) and Tracking Primer SBE Q1 (SEQ ID NO:16) are single and double underlined, respectively. PCR primer Q1 Rev (SEQ ID NO: 17) is shown in the 3′-5′ direction.

FIG. 10 shows signals obtained according to Example 3, comparing signals generated from mutant nucleic acid (a one base insertion compared to normal) in samples obtained from cancer patients to signals generated from normal nucleic acid samples using labeled “A” terminator nucleotide. The wild type (“WT”; SEQ ID NO:18) and mutant (“MT”; SEQ ID NO: 19) sequences being tracked are provided, wherein MT is a one base insertion.

FIG. 11 shows PCR amplicon fragments tiled across a genomic region of interest.

FIG. 12 shows sequencing primers tiled within an amplicon of interest.

FIG. 13 are signals obtained according to Example 1 showing mutant nucleic acid detection in test samples doped with 50%, 25%, 10%, 5%, 2.5%, and 1% mutant nucleic acid constructs compared to a normal (0%) nucleic acid sample using labeled “A” terminator nucleotide. The wild type (“WT”; SEQ ID NO:20) and mutant (“MT”; SEQ ID NO:21) sequences being tracked are provided.

### DETAILED DESCRIPTION

With the discovery of the human genome, there is an increasing need to scan genomic regions for insertions, deletions, rearrangements and point mutations (“mutations”) in heterogeneous DNA samples. Additionally, there are certain diseases that are associated with the presence of a particular mutation (e.g., colorectal cancer). Often, these mutations are present at a relatively low level in a sample relative to the wild type nucleic acid in a sample. In certain embodiments of the invention, the sample is prepared from a specimen selected from the group consisting of stool (including stool homogenate), sputum, pus, blood (including whole blood, blood plasma, and blood serum), urine, cerebrospinal fluid, seminal fluid, saliva, breast nipple aspirate, biopsy tissue, cerebrospinal fluid, and pancreatic fluid. However, any tissue or body fluid specimen may be used according to methods of the invention.

Aspects of the invention relate to methods for detecting the presence of one or more mutations in a region of a mutant nucleic acid that may be present in a biological sample at a low frequency (e.g., about 10%, 5%, 1%, 0.1% or lower) relative to corresponding normal (non-mutant) nucleic acid. Aspects of the invention are useful for detecting low frequency mutations that may occur at one or more positions within a region suspected of being altered (e.g., containing one or more mutations). Such a region may be a mutation hot spot of between 50 bases and 1,000 based in length. For example, a region suspected of containing one or more mutations may be about 100 bases, about 200 bases, about 300 bases, about 400 bases, about 500 bases, about 600 bases, about 700 bases, about 800 bases, or about 900 bases long. However, shorter, longer, or intermediate length regions may be scanned. According to the invention, the presence of one or more mutations in such a region may be difficult to detect using known methods, because mutant nucleic acid molecules may be present at a low frequency in a nucleic acid preparation and also because the position of the one or more mutations within such a mutant nucleic acid region are not known. Aspects of the invention involve performing single base scanning sequencing reactions under conditions that enhance the signal obtained from a mutation within a specific region. For example, mutant signal may be enhanced by increasing the amount of template that is introduced, using a ratio of terminator to extending nucleotides that increases the signal of low frequency events, running relatively short sequencing reactions (e.g., about 100 bases) to increase the signal, using two enzymes with different specificities for terminating and extending nucleotides to reduce the amount of misincorporation that may result in a false positive, performing analysis using appropriate software to quantify the amount of signal (e.g., area under the curve) for each base and quantify the amount of mutant material, or any combination thereof.

In a first step, a method of the invention includes preparing a nucleic acid sample (typically DNA) for scanning. In some embodiments, a nucleic acid preparation may be amplified from a biological sample. To the extent enough nucleic acid exists in the sample, amplification is not required. However, to the extent amplification is desired, any of a variety of methods can be used including, but not limited to PCR, RT-PCR, OLA, rolling circle, single base extension, and others such methods known to one skilled in the art. To the extent PCR is used, primers are designed to amplify a targeted region of a genome or other source of nucleic acid. For example, the region may be a mutation cluster region (“MCR”) or any other region suspected of being associated with a mutation diagnostic for a disease (e.g., mutations present in a gene such as APC, P53, BAT-26, PIK3CA, beta-catenin, or a portion thereof such as exon 9 or exon 20 of the PIK3CA locus, exon 5 of the beta-catenin locus, or a portion of any of the above suspected of containing a mutation). Nucleic acid regions that may contain one or more nucleic acid mutations associated with cancer are described for example in Cancer Research (Mar. 15, 1998) 58, pp 1130-1134, and Science (Apr. 23, 2004) 304(5670): 554, the disclosures of which are incorporated herein in their entirety. However, aspects of the invention may be useful for scanning any genomic region in which one or more mutations may be associated with diseases such as cancer (e.g., a “hotspot” region for mutations associated with diseases such as cancer). If using PCR, a primer pair may be designed to amplify the entire region in one reaction. Alternatively, several primers can be designed to overlap. In this case, two or more sets of primers are used to amplify the region. The sets of primers can be used in separate amplification reactions or in one multiplex reaction.

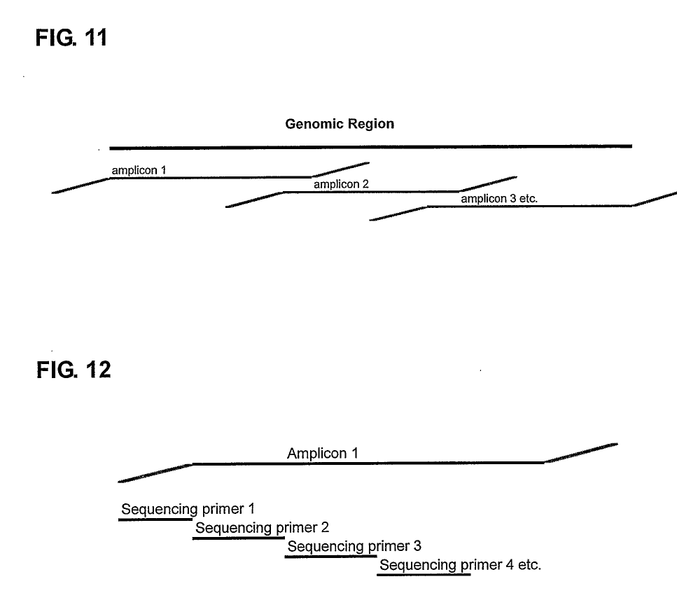

In one embodiment, and referring to FIG. 11, PCR amplification fragments are generated across a genomic region of interest 2 using the following parameters. PCR primers (not shown) are designed such that a 3′ portion of the primer is complementary to the region 2, while a 5′ portion of the primer is not complementary to the region 2. This primer design allows for multiplexing without having to optimize the amplification reaction for each set of primers. The sections of the primers that are not complementary to the region are designed such that the primers have a similar annealing temperature. Based on the sequence of the primers, the annealing temperature can be calculated using methods know in the art and the sequence can be modified to achieve the desired annealing temperature. Alternatively, the PCR primers do not need to have segments that are not complementary to the region (for example, if multiplexing is not desired). The PCR amplification of any one or more of fragments 4, 8, or 10 produced through this reaction overlap. Therefore, mutations occurring within the PCR primer region of one PCR amplification fragment will be picked up by the single base tracking reaction for the adjacent PCR amplification fragment (described below) in the region of the overlap. The amount of overlap will be determined by how close to the tracking primer a mutation can be detected. The amplicons 4, 8, or 10 that are the products of a PCR reaction using primers with a complementary 3′ portion and a noncomplementary 5′ portion will also have the non-complementary sequence 6 at each end.

One of the PCR primers that generates each fragment may be biotinylated if post-PCR cleanup is desired. For example, the biotinylated PCR amplification fragments can be run over a column having complementary streptavidin bound to beads. This removes the amplified fragments from the rest of the nucleic acid in the amplification reactions, simplifying the ability to see relatively low amounts of mutant nucleic acid. The bound amplification fragments are then optionally eluted from the column. Alternatively, other binding partners that are known in the art may be used with one of the partners attached to the PCR primer. Attachment may be by any suitable linkage or linkage method known to one skilled in the art.

Aspects of the invention may involve using a high number of amplification cycles to reach a point at which amplification is saturated in order increase the probability of amplifying any mutant templates that are present in a sample. For example, the number of amplification reactions may be above 30, for example above 40, above 50, about 60 or above. In one embodiment, an entire amplification product may be analyzed in one single base scanning reaction. In other embodiments, an entire amplification reaction may be partitioned into aliquots that are analyzed using two or more different single base scanning reactions (optionally with two or more primers).

If desired, PCR amplification can be performed in the presence of blocking oligonucleotides that suppress amplification of desired sequences in a population of nucleic acid sequences. The sequences whose amplification is blocked are typically those that are present in excess in a starting population of mixed nucleic acids. For example, if a sequence containing a mutation is present in a small amount in a population of nucleic acid sequences that do not contain the mutation, amplification of the latter sequences can be suppressed by adding blocking oligonucleotide or nucleotides prior to, or concurrently with, performing the PCR reaction. The blocking oligonucleotides preferably bind specifically (and in some embodiments, exclusively) to sequences not containing the mutation. The result is to increase the relative representation of the mutant sequence in a population of amplified sequences. A blocking oligonucleotide can be, e.g., a peptide nucleic acid (PNA), a locked nucleic acid (LNA), or a oligonucleotide including one or more phosphine analogues, PEGA modified antisense constructs. 2′-O-methyl nucleic acids, 2′-fluoro nucleic acids, phosphorothioates, and metal phosphonates.

Accordingly, once a nucleic acid preparation is ready (e.g., once amplification fragments or products are collected) a modified single base-tracking reaction is undertaken. Now referring to FIG. 12, tracking primers 12, 14, 16, and 18 are tiled along each amplicon produced in the amplification reaction (“amplicon 1” is shown), and single base tracking reactions are performed. However, different numbers of single base-tracking reactions may be performed (e.g., 1, 2, 3, 4, or more) as the invention is not limited in this respect. Typically, if more than one reaction is performed to analyze a particular nucleic acid region, the tracking primers are spaced about 50 to about 100 nucleotides apart (relative to the target nucleic acid to which they bind). Each tracking reaction includes the four normal dNTP's (deoxynucleotide triphosphate), i.e., dATP, dCTP, dGTP, dTTP, and one terminator nucleotide labeled with a detectable label (e.g., ddNTP (dideoxynucleotide triphosphate) or AcycloNTP (PerkinElmer Sciences, Wellesley, Mass.) where the base of the terminator nucleotide is a single base chosen from A, C, G and T). Up to four single base tracking reactions, one for each terminator base, are conducted. In each reaction, a different one of the terminator nucleotides (having one of the bases A, C, G, T) is used per tracking primer. In a preferred embodiment, the terminator nucleotide is labeled. These labels can be dyes or other chemical compounds that fluoresce when exposed to the proper wavelength of light. Labeled and/or unlabeled nucleotides also may be synthesized with mass tags to increase fragment separation and resolution if detection is for example by mass spectroscopy. The result of the single base tracking reaction is a series of nested fragments ending at the base corresponding to the labeled terminator nucleotide. Alternatively, rather than labeling the terminator nucleotides, the dNTP having the same base as the terminator nucleotide being used can be labeled or the primer itself can be labeled. The choice of labeling method is within the skill of the artisan working in the art field.

In one aspect, base-tracking reactions may be performed with a ratio of extending nucleotides to terminating nucleotides of about 50:1 (for example a ratio of about 12.5:1 for each individual extending nucleotide to the terminating nucleotide). However, lower ratios may be used provided that the rate of misincorporation of the labeled nucleotide does not exceed the level of mutant nucleic acid that is being assayed for. Ratios of extending nucleotides to terminating nucleotides may be between about 10:1 and about 100:1 (e.g., about 10:1, 20:1, 30:1, 40:1, 50:1, 60:1, 70:1, 80:1, 90:1, or an intermediate value). However, other ratios may be used as the invention is not limited in this respect. Typically, a ratio of extending nucleotides to terminating nucleotides may be lower than one used in a standard sequencing reaction but higher than one used in a standard primer extension reaction (e.g., single base extension reaction) designed to detect a specific mutation. A ratio may be chosen so that the single base-scanning reaction extend for between about 20 to about 150 bases, for example for between about 50 and about 100 bases so that any low frequency genetic event produces sufficient signal to be detected and/or quantified.

In certain aspects of invention, primer extension may be performed using two or more different polymerases for detecting and identifying mutant nucleic acids within a heterogeneous population of nucleic acids. In one embodiment, methods of the invention reduce misincorporation of terminator nucleotides in single base-scanning reactions by providing a first polymerase that preferentially incorporates extending nucleotides and a second polymerase that preferentially incorporates terminating nucleotides. In one embodiment, one or both of the polymerases misincorporate(s) an incorrect nucleotide (e.g., a terminating nucleotide) with a frequency of less than about 10%, for example less than about 5%, less than about 1%, or less than about 0.1%. It should be appreciated that misincorporation of a terminating nucleotide at a non-complementary position in an extension reaction can generate a false positive indication of the presence of a mutation at the site of misincorporation. According to the invention, a significant source of misincorporation may result from a correctly hybridized primer being extended with an incorrect nucleotide (i.e. a nucleotide that is not complementary to the template at the position where it is incorporated into the primer extension product).

In one aspect, misincorporation may be reduced by performing a single base-scanning reaction in the presence of at least two polymerases, each of which preferentially incorporates one of two nucleotides: an extending nucleotide or a terminating nucleotide. By including different polymerases for the extending and terminating nucleotides, polymerases that have low frequencies of incorrect base incorporation may be used. Preferably, one of the nucleotides is labeled and the other one is not labeled. The labeled nucleotide is preferably the one that is incorporated in the primer extension product when the template contains a sequence to be detected. In alternative embodiments, both first and second nucleotide may be labeled if they are differentially labeled such that the label on one of the nucleotides is detectably different from the label on the other nucleotide.

According to the invention, reducing the rate of misincorporation increases the sensitivity of single base scanning assays and allows for the detection of rare nucleic acid variations in heterogeneous biological samples. Misincorporation of a nucleotide corresponding to a mutation in a primer extension reaction can lead to a false positive detection of the presence of the mutation in a nucleic acid sample. In a typical primer extension reaction on a biological sample that contains mostly wild-type nucleic acids, misincorporation results in a background level of false positive signal that is high enough to obscure a true positive signal generated from a relatively small amount of mutant nucleic acids. Therefore, by reducing the amount of misincorporation, aspects of the invention provide highly sensitive and highly specific assays for detecting rare variant and/or mutant nucleic acid in heterogeneous biological samples.

In one aspect of the invention, single base scanning methods include identifying a target nucleic acid region suspected of containing a variation, and interrogating the target region using a single base scanning reaction. A primer is hybridized to a single stranded nucleic acid in the presence of extension nucleotides and polymerases, and the primer is extended through the target region creating primer extension products that are subsequently detected and/or quantified.

As discussed herein, different polymerases characterized by preferential nucleotide incorporation may be used to increase the signal to noise ratio for detecting low frequency events (e.g., mutant nucleic acids that are present at a low frequency in a biological sample containing an excess of non-mutant nucleic acids). In one embodiment, a first polymerase preferentially incorporates the extending nucleotide that is used, and a second polymerase preferentially incorporates the terminating nucleotide that is used. Preferential incorporation of one nucleotide over another can be measured using any suitable technique, for example by running parallel reactions which differ only in the type and concentration of each nucleotide. The relative incorporation efficiency of two different nucleotides can be reflected in the concentrations of nucleotides in the two reactions. For example, a first reaction that contains 10-fold more of a first nucleotide than a second reaction containing a second nucleotide demonstrates that insertion of the first nucleotide is 10-fold less efficient than that of the second nucleotide. These concentration levels can be measured by various methods, including, for example, performing titration assays, running the samples on DNA sequencing gels and visualizing the extent of nucleotide incorporation by autoradiography, running capillary electrophoresis assays and determining the levels of nucleotide incorporation. In one embodiment, if the incorporation of a first nucleotide over a second nucleotide is a 10-fold or greater difference, a determination can be made that the first terminator was preferentially incorporated. Preferably, a first polymerase incorporates a first nucleotide with between a 2 fold and a 100 fold preference. In certain embodiments, the ratio of incorporation may be between 5 fold and 50 fold, for example the ratio may be about 10 fold. However, ratios of less than 2 fold and greater than 100 fold can also be useful. The preference of the first polymerase for the first nucleotide relative to the second nucleotide can also be measured as a percentage increase in incorporation in an assay. Preferably, a first polymerase has between about a 5% and a 100% preference for a first type of nucleotide relative to a second type of nucleotide, for example between about 25% and about 75%. Examples of DNA polymerases with preferential nucleotide incorporation properties include those that preferentially incorporate dideoxy terminators over acyclic-terminators include Taq polymerase and Thermo Sequenase. (Gardner, A., (2002) “Acyclic and dideoxy terminator preferences denote divergent sugar recognition by archaeon and Taq DNA polymerases”, Nucleic Acids Research, Vol. 30, No. 2 pp. 605-613.) Examples of DNA polymerases that preferentially incorporate acyclic terminators include Vent, Vent A488L, Deep Vent, 9°Nₘ, Pfu, and AcycloPol. In one embodiment of the invention, preferential incorporation can also be achieved by using dye-labeled terminators. Examples of DNA polymerases that preferentially incorporate dye-labeled terminators include Vent DNA polymerase, which preferentially incorporates dye-labeled dCTP analogs over unmodified dCTPs, and dye-acyCTPs over dye-ddCTPs, and Vent, Deep Vent, Pfu and 9°Nₘ polymerases, which preferentially incorporate dye-acyNTPs over dye-ddNTPs. Similarly, preferential incorporation of terminator nucleotides relative to extending nucleotides or extending nucleotides relative to terminator nucleotides may be assessed and polymerases with preferential incorporation properties may be used as described herein.

The products of the single base tracking reactions (e.g., from each amplification fragment) may be separated using electrophoresis, e.g., CAGE (capillary gel electrophoresis). The shorter fragments elute from the gel first while the longer fragments elute from the gel last. As the fragments are eluted from the gel, the signal from the label is detected and a pattern of signals for all the fragments is determined (examples of patterns are shown in the Examples below). For example, if fluorescent labels are used, an ABI 3100 DNA Sequencer can be used to read out the signal pattern. Each reaction can be analyzed independently and/or multiplexed.

In one embodiment, the same single base tracking procedure may be carried out for a known normal nucleic acid sequence (e.g., a wild type sequence). The signal pattern generated for the normal sequence may be compared with the signal pattern from the sample. Insertions, deletions and point mutations may be identified by a change in the peak pattern relative to the wild type peak pattern. This comparison can be undertaken manually or in an automated fashion. For example, comparisons may be performed using software that quantifies the amount of different types of signals at different positions (e.g., for extension products of different lengths). The amount of a signal at a position that is not expected to have a signal in a wild-type template may be used to determine a frequency of mutant or variant nucleic acids in a biological sample. This modified sequence reaction produces results that can detect a mutant to wild type ratio in a sample of less than 1:1. For example, the invention can detect mutations present at a ratio of about 1:4 (mutant to wild type), about 1:10 (mutant to wild type), about 1:100 (mutant to wild type), or less than about 1:100 (mutant to wild type), for example about 1:1,000 or less. Accordingly, this tracking method has a higher sensitivity for mutations than do current sequencing reactions.

Additionally, the single base tracking reactions described above can be run in the forward and reverse directions. For example, if the procedure described above was the “forward” direction, primers would be designed to sequence the amplification fragments in the opposite direction. This reverse direction single base tracking is a manner to confirm that the result obtained in the forward direction is accurate. The single base tracking reaction can be run in the forward and/or reverse directions as many times as is desired to confirm the results.

Certain aspects of the invention are illustrated by the following examples. However, the following examples are not limiting and other aspects of the invention are described herein.

### EXAMPLES

### Example 1

The following is an example of an experimental construct designed to illustrate a method for detecting a low frequency mutation in a heterologous population of sequences using a known sequence contained in a plasmid. In this experiment, it was shown that a single nucleotide mutation comprising as little as 1% of the sample was detected.

### PCR

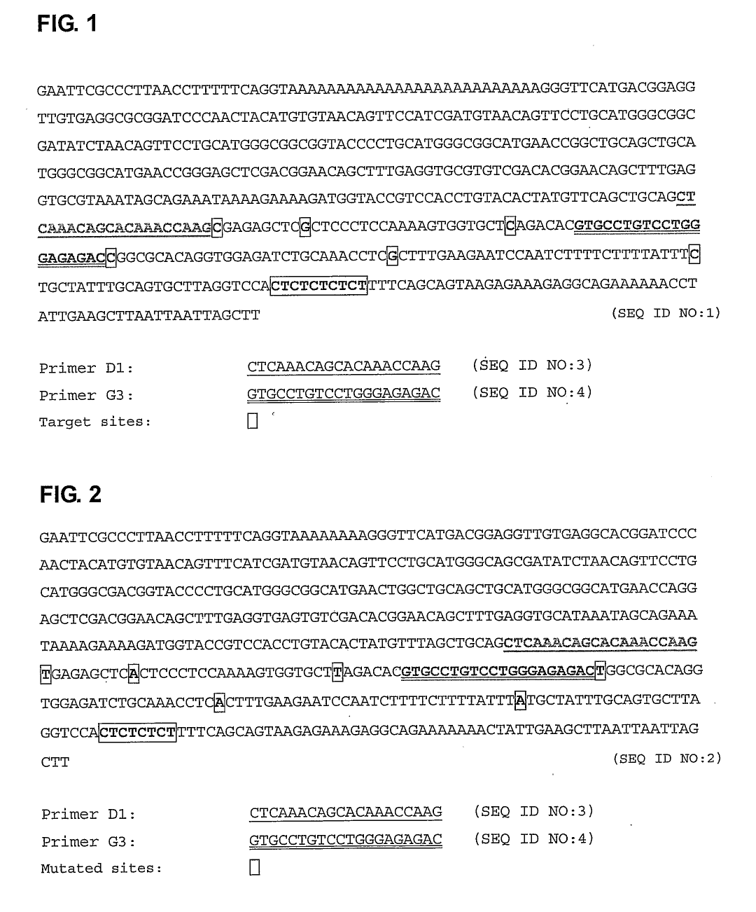

Separate, single PCR was performed on both a normal “wild type” plasmid and a corresponding mutant plasmid, in order to amplify the normal DNA sequence in the wild type plasmid (FIG. 1, whole plasmid not shown) and the mutated region from the mutant plasmid (FIG. 2, whole plasmid not shown). FIG. 1 shows the nucleic acid sequence of the fragment amplified for the normal plasmid, with tracking primer sequences and polymorphic sites as marked. FIG. 2 shows the nucleic acid sequence of the fragment amplified from the mutant plasmid, again with tracking primer sequences and mutation sites as marked.

Biotinylated primers were chosen such that the amplified fragments had a biotinylated end that was captured on a column having beads and streptavidin on the beads. Briefly, 50 μl of PCR reaction product was added to 200 μg of Streptavidin beads (Dynal) and was incubated for 15 minutes. One-tenth molar NaOH was added for 5 minutes to elute off the complementary (non-bound) strand. A wash with buffer and final elution with TE followed. The amplification fragments of interest remain bound to the beads. Typically, 5 μl of these beads are added to the single base tracking reactions. In this instance, in order to generate the experimental construct, the capture amplified fragments were then combined with wild type fragments in varying amounts such that the amplified mutant fragment would be combined with the wild type fragment to form a sample containing 1% mutation, 2% mutation, or 4% mutation.

### Tracking Reactions

Once the mutant sample was combined with the normal sample, single base tracking reactions were carried out for the 1%, 2%, and 4% mutant samples as well as for a pure wild type sample as follows. One microliter of 5 μM HPLC purified stock primer, 5 μl of amplified DNA, 0.5 μl each of AcycloPol (Perkin Elmer Life Sciences, Wellesley, Mass.) and ThermoSequenase® (Amersham Pharmacia Biotech, Buckinghamshire, England) buffers, 0.025 μl AcycloPol Enzyme (22 units/50 μl), 0.05 μl ThermoSequenase Enzyme (32 units/μl), 0.25 μl of a 0.025 mM stock of each unlabeled dNTPs (A, T, G, and C) for a total of 1 μl, 0.1 μl R110-AcycloNTP (only one terminator base per reaction, the base chosen from A, C, G and T; 0.1 mM stock diluted 1/20) (Perkin Elmer Life Sciences) (terminator nucleotide labeled with a detectable dye that fluoresces), and 1.9 μl of water, are combined. The steps for the cycling conditions were step 1: 95 degrees Celsius for one minute, step 2: 95 degrees Celsius for 30 seconds, step 3: 52 degrees Celsius for 10 seconds, and step 4: 72 degrees Celsius for 10 seconds. The mixture is then thermocycled through steps 2 through 4 for 30 cycles. At the completion of cycling, the mixture is treated with 1 μl of shrimp alkaline phosphatase at 37 degrees Celsius for 30 minutes. The reactions were conducted with the primers D1 or G3 (locations shown in FIG. 1) with labeled “A” terminator nucleotide, and with primer G3 with labeled “T” terminator nucleotide.

### Electrophoresis

Nested fragments produced by the single base tracking reaction are then put into an electrophoretic analyzer. The fragments are first separated by size using capillary electrophoresis. Next, the chemical labels attached to the fragments are excited and the intensity and location of the fluorescent signals from fragments are identified and recorded by the analyzer. The location and size of the peaks produced by the mutant nucleic acid samples were then compared to the location and size of the peaks produced by wild type or normal samples in order to determine the existence and location of a mutation or the sequence of mutant nucleic acids. In this manner, it was possible to analyze nucleic acid test samples of a genomic region and compare the location and size of peaks produced by labeled nucleic acid fragments to peaks produced by labeled nucleic acid fragments from a known non-mutant region of the same nucleic acid. By comparing the size and location of the labeled nucleic acid fragments from the known normal sample to those of the test sample, it is possible to determine if different signal peaks exist which would be indicative of a mutation.

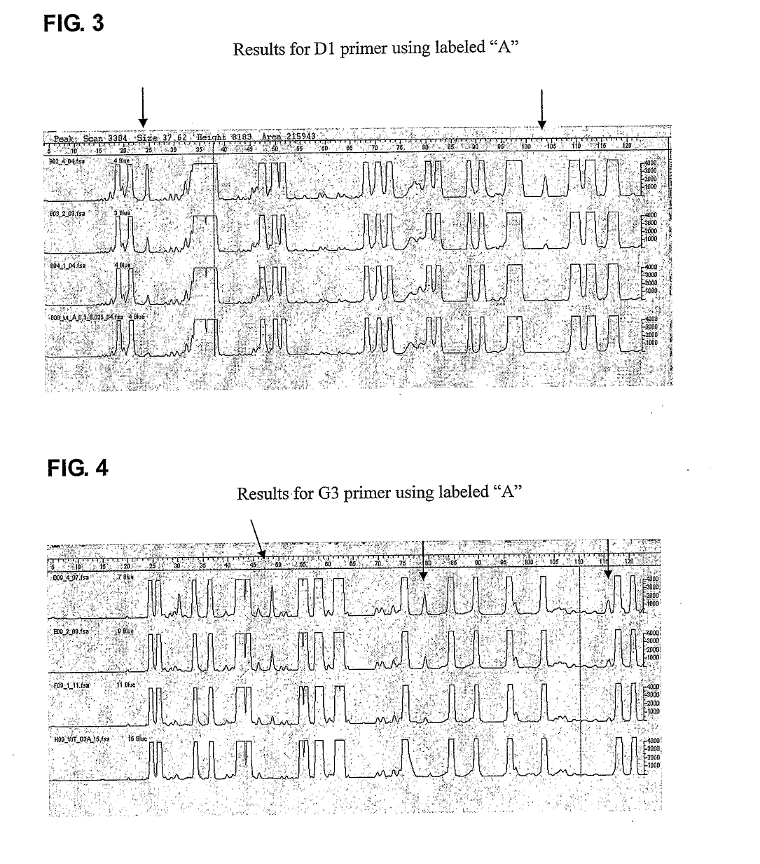

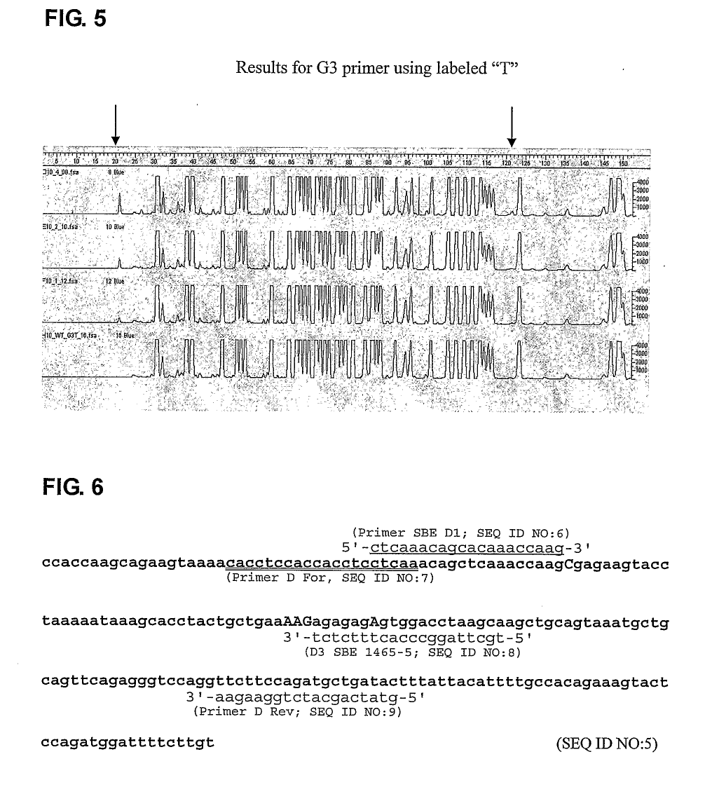

In order to analyze the tracked samples, one microliter of the completed single base tracking reaction mixture was injected into an Applied Biosystems® 3100 Genetic Analyzer (Applied Biosystems, Foster City, Calif.) for capillary electrophoresis and signal analyzation. FIG. 3 shows the results when primer D1 is used along with labeled “A” terminator nucleotide. The fragment size is illustrated on the X-axis (shorter fragments on the left and longer fragments on the right), while the signal intensity is shown on the Y-axis. Shown are samples doped with 4%, 2%, and 1% mutant sequences, and one wild type sequence for comparison. The arrows indicate areas to be compared between the mutant samples and the wild type sample. The peaks corresponding to signals allow detection of the point mutations in all mutant samples because the normal control sample does not have corresponding peaks. FIG. 4 shows the results when primer G3 is used along with labeled “A” terminator nucleotide. Shown are samples doped with 4%, 2%, and 1% mutant sequences and one wild type sequence for comparison. The arrows indicate the areas to be compared between the mutant samples and the wild type samples (50, 80, and 115 bases). The peaks corresponding to signals allow detection of the mutations because the normal sample does not have corresponding peaks. FIG. 5 shows the result when primer G3 is used along with labeled “T” terminator nucleotide. Shown are samples doped with 4%, 2%, and 1% mutant plasmid sequences and one normal sequence for control. The arrows indicate the areas to be compared between the point mutant samples and the wild type sample. The peaks corresponding to signals allow detection of the mutations because the normal sample does not have corresponding peaks. FIGS. 3-5 show that several tracking primers were tested and 1% mutant DNA was detected up to 100 bases from the primer site for several different primers. This is a five to ten fold increase in sensitivity over current methods known in the art.

### Example 2

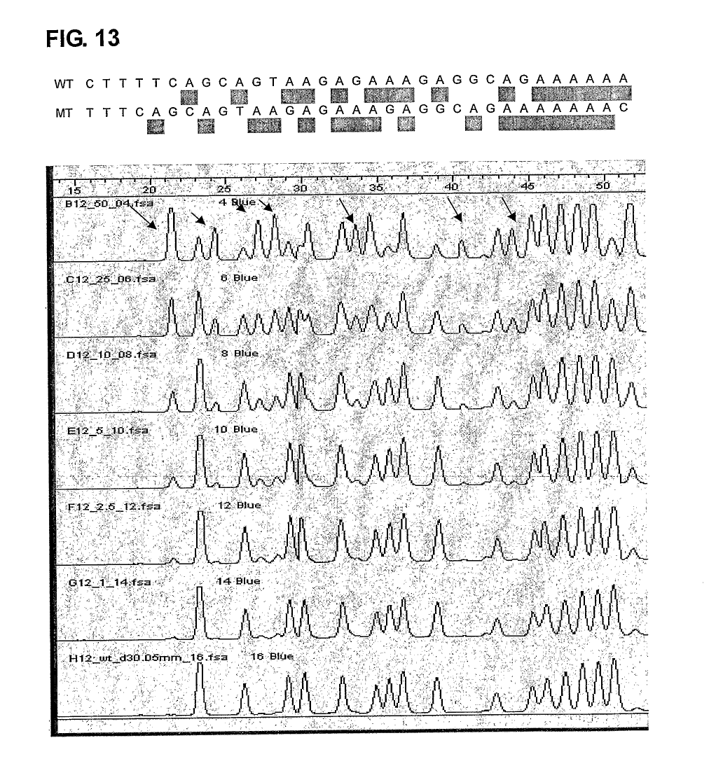

The experiment in this example was performed in the same manner as Example 1 except this illustrates a two (2) base deletion mutant. The samples were sequenced using dATP, dCTP, dTTP, and dGTP, with labeled “A” terminator nucleotide. The relevant portions of the mutant and wild type plasmids are shown in FIG. 13. The boxes below the sequences indicate the positions at which the single base tracking reaction can be terminated by a labeled terminator nucleotide. The samples were then analyzed electrophoretically as in Example 1. FIG. 13 shows the sequence results of sample populations of nucleic acids containing various ratios of mutant and wild type construct. The amount of doped mutant sequence ranged from 50% down to 1%. Arrows indicate the areas to be compared between the mutant samples and the wild type sample. The peaks corresponding to signals allow detection of the mutation in the mutant sample because the wild type sample does not have corresponding peaks.

### Example 3

This example is the same as Example 1, except sample DNA obtained from stool samples in cancer patients with known mutations was used instead of plasmid nucleic acid sequences. This example amplified a genomic region of interest and subsequently used tracking primers designed to span across the PCR region. Known regions of the mutation cluster region (MCR) of the adenomatous polyposis coli (APC) gene were used to demonstrate the applicability and sensitivity of one aspect of the invention on mutations in genomic DNA. As in Example 1, mutant sample presented peaks not present in wild type samples that indicate the presence of a mutation. This example shows that comparing the peaks or a mutation associated with cancer with a known normal can be used to screen for a disease.

PCR for the APC-MCR region of interest was carried out as in Example 1 using samples from two cancer patients known to contain a mutation in the region of interest and two normal samples. FIG. 6 shows the nucleic acid sequence for a region of APC codon (1450-1465). PCR primer sites are identified with a single underline, while tracking primer sites are identified with a double underline. Additionally, D1 is a sense primer, and D3 is an antisense primer.

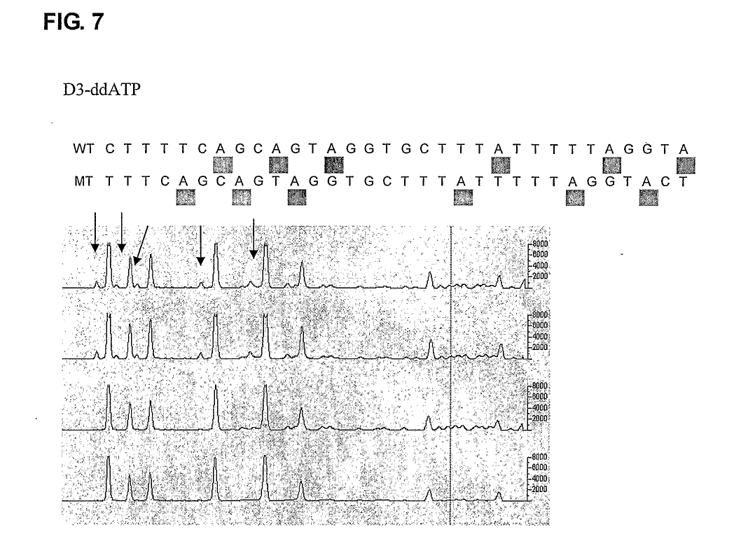

FIG. 7 shows the signals generated by single base tracking reaction products from the D3 primer reacted with labeled “A” terminator nucleotides. Above the signals generated by the single base tracking reaction products, the relevant wild type and mutant nucleic acid sequences are listed with boxes indicating the positions at which single base tracking reactions can be terminated. The mutation is a two base deletion. The top two signal patterns represent nucleic acid from cancer patients, while the bottom two samples are signal patterns from normals. The arrows indicate the areas to be compared between the cancer patient samples and the normal samples. The peaks corresponding to the deletion mutation are present in the nucleic acid from the cancer patients but are not found in the nucleic acid from normal DNA. In addition, the positions of the peaks after the deletion in the mutant sequence are shifted in position to the left (shorter) as compared to the control.

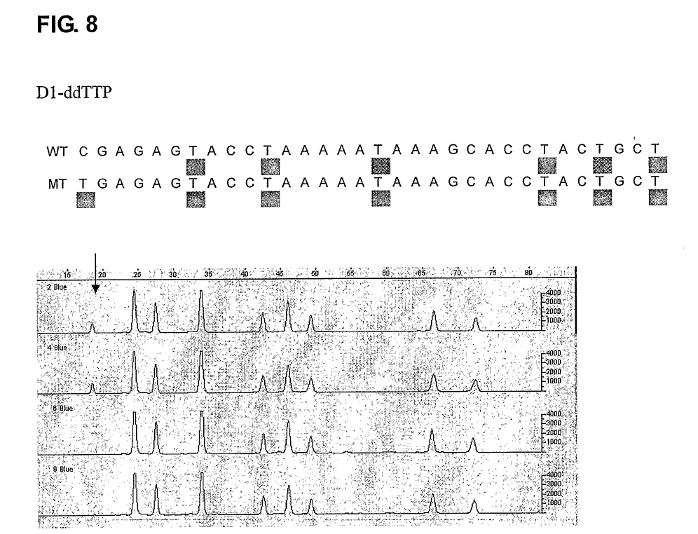

FIG. 8 shows the signals generated by single base tracking reaction products from the D1 primer reacted with labeled “T” terminator nucleotides. Above the signals generated by the single base tracking reaction products, the relevant wild type and mutant nucleic acid sequences are listed with boxes indicating the positions at which the single base tracking reactions can be terminated. The mutation is a point mutation. The area to be compared is identified by the arrow. The peak corresponding to the point mutation is present in the nucleic acid from the cancer patients but not from the normals. The lower two signal patterns are from normal samples.

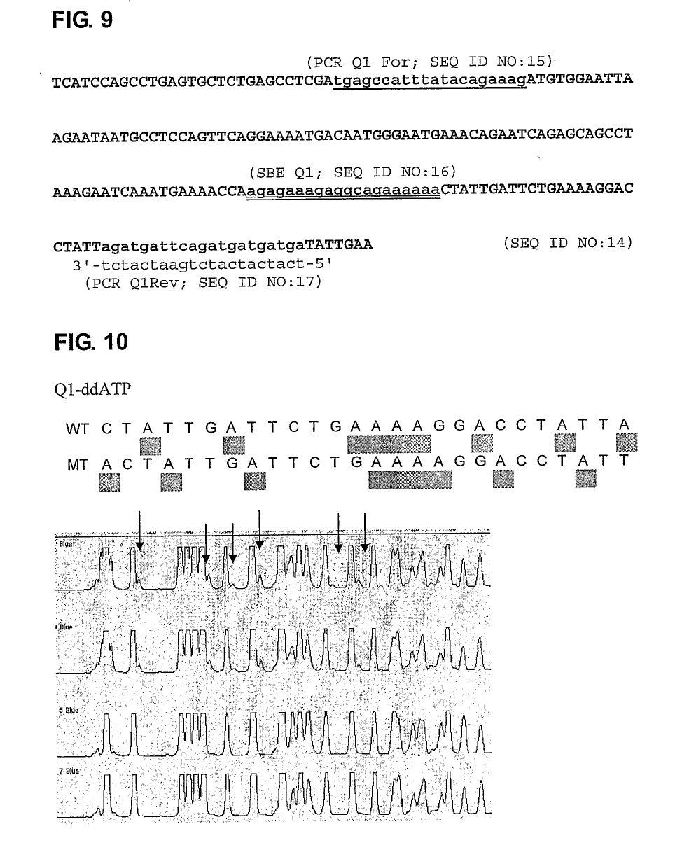

FIG. 9 shows the nucleic acid sequence of APC coding region from residues 1503-1573, wherein the residue at position 1554 is targeted for interrogation, with PCR primer regions underlined, and the tracking primer region double underlined. The assay was conducted as described above. FIG. 10 shows the results from the Q1 tracking primer reacted with labeled “A” terminator nucleotide. Above the single base tracking reaction products are the relevant wild type and mutant nucleic acid sequences with boxes indicating the positions at which the single base tracking reactions can be terminated. The mutation is a one base insertion. The areas to be compared are identified by arrows. The peaks corresponding to the mutation in these areas are present in the cancer patients (top two traces) but are not present in the normal patients (bottom two traces). In addition, the positions of the peaks after the insertion in the mutant sequence are shifted in position to the right (longer) as compared to the control.

### Example 4

This example illustrates obtaining DNA from a stool sample. A stool sample is collected and may be stored at −80° C. before use. The sample is thawed and resuspended in buffer, for example 10 mM Tris-Cl pH8.0, 1 mM EDTA and 150 mM NaCl, or other suitable buffer as known to those of ordinary skill in the art. The sample is then homogenized utilizing an EXACTOR stool shaker (EXACT Laboratories Marlborough, Mass.). Following homogenization, the stool sample is centrifuged to remove all particulate matter, and the supernatants are incubated at 37° C. Proteinase K (0.5 μg/μL) and SDS (0.5%) may be added at this point. The DNA is extracted from the supernatant using Tris saturated phenol (Gibco/BRL, Grand Island, N.Y.), phenol/chloroform/isoamyl alcohol (25:24:1), and chloroform. The DNA is then precipitated (1/10 volume 3M NaAc and an equal volume isopropanol), removed from solution by centrifugation, and resuspended in TE (0.01M Tris pH 7.4, 0.001M EDTA) buffer containing RNase A (2.5 μg/mL), or other suitable buffer.

The following example illustrates a method for preparing a DNA sample from a stool sample, see for example US Published Application No. 20040043467 and US Published Application No. 20040014104, the entire contents of which are incorporated herein by reference.

In aspects of the invention, a large amount of sample may be processed in order to increase the confidence level of isolating or capturing a rare event indicative of very early stage disease (e.g., an adenoma, an early stage cancer, etc.). For example, about 10 g, about 20 g, about 30 g, about 40 g, about 50 g, about 60 g, about 70 g, about 80 g, about 90 g, about 100 g, about 150 g, about 200 g, or more stool sample may be processed using a capture technique described herein.

### EQUIVALENTS

Having described certain embodiments of the invention, it will be apparent to those of ordinary skill in the art that other embodiments incorporating the concepts disclosed herein be used without departing from the spirit and scope of the invention. In particular, it is contemplated by the inventors that various substitutions, alterations, and modifications may be made to the invention while remaining within the scope of the invention as defined by the claims. The described embodiments are to be considered in all aspects as only illustrative and not restrictive.

What is claimed is:

1. A method for detecting a difference between two nucleic acids, the method comprising:
extending a first primer complementary to a target nucleic acid in the presence of a deoxynucleotide mixture and a terminator nucleotide to produce at least one product, wherein the terminator nucleotide comprises a detectable label;
detecting a signal from the at least one product; and
comparing the signal from the at least one product with a signal generated from a comparison nucleic acid in substantially the same manner as the signal is generated from the target nucleic acid and wherein a difference between the signals indicates at least one difference between the target nucleic acid and the comparison nucleic acid.

2. The method of claim 1 further comprising the step of amplifying a nucleic acid to form the target nucleic acid.

3. The method of claim 1 wherein the extending step comprises extending the primer in the presence of dATP, dCTP, dGTP, and dTTP.

4. The method of claim 1 wherein the target nucleic acid comprises a nucleic acid suspected of containing a mutation.

5. The method of claim 1 wherein the extending and comparing steps are repeated.

6. The method of claim 5 wherein the steps are conducted at least four times, each time using a different one of A, C, G, or T for the base of the terminator nucleotide.

7. The method of claim 1 further comprising extending a second primer complementary to the target nucleic acid in the presence of the deoxynucleotide mixture and the terminator nucleotide to produce at least one secondary product;
detecting a signal from the at least one secondary product; and
comparing the signal from the at least one secondary product with a signal generated from a comparison nucleic acid in substantially the same manner as the signal is generated from the target nucleic acid and wherein a difference between the signals indicates at least one difference between the target nucleic acid and the comparison nucleic acid.

8. The method of claim 1 further comprising extending a second primer complementary to a strand complementary to the target nucleic acid in the presence of the -deoxynucleotide mixture and the terminator nucleotide to produce at least one secondary product;
detecting a signal from the at least one secondary product; and
comparing the signal from the at least one secondary product with a signal generated from a comparison nucleic acid in substantially the same manner as the signal is generated from the target nucleic acid and wherein a difference between the signals indicates at least one difference between the target nucleic acid and the comparison nucleic acid.

9. The method of claim 1 wherein the comparison nucleic acid comprises a wild type nucleic acid.

10. The method of claim 11 wherein the signal from the comparison nucleic acid is determined prior to the signal from the target nucleic acid.

11. The method of claim 1 wherein the signal comprises a fluorescent light emission.

12. A method for detecting a difference between two nucleic acids, the method comprising:
extending a first primer complementary to a target nucleic acid in the presence of a deoxynucleotide mixture comprising at least one detectable label and a terminator nucleotide to produce at least one product
detecting a signal from the at least one product; and
comparing the signal from the at least one product with a signal generated from a comparison nucleic acid in substantially the same manner as the signal is generated from the target nucleic acid and wherein a difference between the signals indicates at least one difference between the target nucleic acid and the comparison nucleic acid.

13. A method for detecting a difference between two nucleic acids, the method comprising:
extending a first primer, the first primer comprising a detectable label and wherein the primer is complementary to a target nucleic acid, in the presence of a deoxynucleotide mixture and a terminator nucleotide to produce at least one product;
detecting a signal from the at least one product; and
comparing the signal from the at least one product with a signal generated from a comparison nucleic acid in substantially the same manner as the signal is generated from the target nucleic acid and wherein a difference between the signals indicates at least one difference between the target nucleic acid and the comparison nucleic acid.

14. A method for detecting a difference between two nucleic acids, the method comprising:
extending a first primer complementary to a target nucleic acid in the presence of a deoxynucleotide mixture and a terminator nucleotide to produce at least one product;
detecting a mass of the at least one product; and
comparing the mass of the at least one product with a mass that is generated from a comparison nucleic acid in substantially the same manner as the mass is generated from the target nucleic acid and wherein a difference between the masses indicates at least one difference between the target nucleic acid and the comparison nucleic acid.

15. The method of any one of claims 1-14, wherein the target nucleic acid is selected from the group consisting of genomic DNA, complementary DNA (cDNA) and RNA.

16. The method of any one of claims 1-15, wherein the at least one difference between the target nucleic acid and the comparison nucleic acid indicates the presence of a mutation in the target nucleic acid.

17. The method of claim 16, wherein the presence of the mutation in the target nucleic acid is an indicator of a disease.

18. The method of any one of claim 1-15, wherein said target nucleic acid and comparison nucleic acid are contacted with a blocking oligonucleotide prior to extending said first primer.

19. The method of claim 18, wherein said blocking oligonucleotide is a peptide nucleic acid (PNA).

20. The method of claim 1, wherein two polymerases are used.

21. The method of claim 20, wherein each polymerase preferentially incorporates a different nucleotide type.

22. The method of claim 21, wherein an acyclic terminator, and a polymerase that preferentially incorporates the acyclic terminator are included in the reaction.
